(19) United States
(12) Patent Application Publication
(10) Pub. No.: US 2005/0124530 A1
(43) Pub. Date: Jun. 9, 2005

(54) Fragrance compositions

(76) Inventors: Serge Creutz, Rocourt (BE); Sylvie Bouzeloc, Nivelles (BE)

Correspondence Address: DOW CORNING CORPORATION CO1232, 2200 W. SALZBURG ROAD, P.O. BOX 994, MIDLAND, MI, 48686-0994, US

(21) Appl. No.: 10/507,648

(22) PCT Filed: Mar. 27, 2003
(86) PCT No.: PCT/EP03/03308

(30) Foreign Application Priority Data
Apr. 3, 2002 (GB) 0207647.9

(51) Int. Cl. A61K 7/46

(52) U.S. Cl. 512/3

(57) ABSTRACT

A composition for controlled release of fragrance comprises a blend of a fragrance composition and a waxy hydrophobic material. The way hydrophobic material is a mixture of a wax and a liquid silicone compatible with the wax.

This invention relates to fragrance compositions suitable for incorporation in cleaning products such as laundry detergents, household cleaning products including hard surface cleaners and polishes, fabric softeners, hair shampoos and soaps and shower gels for personal washing, in other personal products such as antiperspirants or deodorants, or in air fresheners or tumble drier sheets.

Fragrances are frequently incorporated in detergents and other cleaning products to give a pleasant odour during use of the cleaning product and to mask the inherent smell of the soap or other surfactant present in the cleaning product. The fragrances are generally complex mixtures of fragrant compounds of varying volatility. Upon storage in a cleaning composition, perfumes and fragrances can be altered through interactions and/or reactions with the other components of the composition. Due to their volatile nature, the fragrant compounds tend to be dissipated with time, particularly the most volatile compounds which are often associated with perceived freshness. Moreover, when used, such as during washing of fabrics with a laundry detergent, most of the perfume is also lost in the aqueous phase during the washing cycle. It has been recognised as desirable that the fragrance should survive storage in the cleaning composition and also survive the cleaning process and should be deposited on the fabric, so that fabrics laundered with a detergent containing the fragrance should have the pleasant odour of the fragrance.

Furthermore, once adsorbed onto the targeted surface, for example fabrics or hair or skin, the fragrance tends to be dissipated very quickly. There is thus a need to improve the storage stability of perfumes and fragrances, their delivery in the application and their long-lasting effect through sustained delayed release once applied on fabrics.

Various methods of protecting the fragrance composition have been proposed. The perfume may be mixed with a porous carrier such as zeolite and then coated with a protective barrier, for example a sugar derivative before incorporation in a laundry detergent as described in WO98/41607. U.S. Pat. No. 4,973,422 describes encapsulating perfume particles with a pH sensitive coating comprising an acrylic resin and cellulose esters. WO-A-98/28936 describes mixing the perfume with an aqueous slurry of polymer beads made of hydrophobic polyacrylate; polyvinyl alcohol can be adsorbed at the surface of the beads to improve deposition. WO-A-00/02981 describes reacting a perfume component with an amine to obtain a release of the active component over a longer period of time.

U.S. Pat. No. 6,050,129 relates to a process for testing diffusivity, odour character and odour intensity of a fragrance material used in an air freshener and describes mixing perfume with a hydrophobic wax such as candelilla wax or carnauba wax and emulsifying the blend in water, preferably with cationic surfactants, to form a long lasting fragrance composition for use in a hair care composition such as a shampoo/conditioner.

WO-A-01/25389 describes a domestic care product comprising a fragrance particle. The particle comprises a fragrance composition and a silicone polymer having a melting point of at least 10° C. At least 20% of the silicone atoms in the silicone polymer have a substituent of 16 carbon atoms or more.

GB-A-1587122 describes fabric conditioning compositions containing particles comprising a perfume and a water-insoluble non-cationic organic carrier, e.g. a wax.

EP-A-539025 describes spray dried composite fragrance microcapsules for fabric conditioning comprising particles of an emulsifiable mixture of wax material and a perfume composition.

U.S. Pat. No. 5,506,201 describes producing fragrance-containing solid particles for incorporation into laundry detergent by melting a fat component such as a fatty acid glyceride, melting a nonionic surfactant with an HLB of 4.3 to 8.6 and combining the melts with an aroma chemical to form a mixture which is rapidly cooled to form a solid material.

EP-A-908174 describes a fragrance composition comprising ellipsoidal hydrophobic particles consisting of a single phase solid solution of a hydrophobic polymer or wax of melting point 35-120° C. having dissolved therein a hydrophobic fragrance material, and a hydrophilic surfactant proximate to the outer surface of the particles.

According to the present invention a composition for controlled release of fragrance comprising a blend of a fragrance composition and a waxy hydrophobic material, characterised in that the waxy hydrophobic material is a mixture of a wax and a liquid silicone compatible with the wax.

The fragrance composition may be solid or liquid and may be a single fragrant compound, or a natural scented oil, or may be a mixture of fragrant compounds and/or natural oils. Examples of such natural oils and fragrant compounds are described in WO-A-01/25389. The fragrance composition can alternatively comprise a chemically protected fragrance compound such as a reaction product of the fragrance compound.

The wax can be an organic wax containing no silicon atom in its molecule such as microcrystalline wax, paraffin wax or a mixture thereof, a long chain fatty acid or a waxy ester thereof such as a triglyceride, for example glyceryl tristearate, a monoester such as octadecyl hexadecanoate, a diester such as ethylene glycol distearate or a tetraester such as pentaeryhthritol tetrastearate, or a long chain fatty alcohol, a long chain fatty amine, a long chain fatty amide, an ethoxylated fatty acid or fatty alcohol, a long chain alkyl phenyl or polyethylene wax. In general the long chain of the fatty acid, alcohol, amine or amide is an alkyl group of at least 12 and preferably at least 16 carbon atoms.

The wax can alternatively be a polysiloxane containing hydrocarbon substituents having 12 or more carbon atoms. The polysiloxane is preferably a polydiorganosiloxane comprising methyl alkyl siloxane units ((CH3)(R′)SiO2/2), where R′ is a long chain alkyl group having 12 or more, preferably 16 to 100 carbon atoms, optionally together with dimethyl siloxane units or units of the formula ((CH₃)(R″)SiO_{2/2}) where R″ is an alkyl group having 1-11 carbon atoms, for example ethyl, a cycloalkyl group such as 2-cyclohexylethyl, a haloalkyl group or an aromatic group. The long chain alkyl group R′ can optionally be substituted by polar substituents such as amino, amido, alcohol, alkoxy, or ester groups. The methyl group of the above siloxane units could be replaced by ethyl or another lower alkyl group if desired. Preferably at least 20% of the silicon atoms in the polysiloxane, and most preferably at least 50%, have an alkyl substituent having 16 to 100 carbon atoms, most preferably 20 to 36 carbon atoms. The polysiloxane may be linear or may be branched, for example it may contain CH3SiO3/2 units or R′SiO_{3/2} units. Alternatively the polysiloxane can be cyclic, for example a cyclic polysiloxane containing 4 or 5 methyl alkyl siloxane units in which the said alkyl group has 16 to 100, most preferably 20 to 36, carbon atoms. One preferred type of wax contains aromatic groups, for example aryl groups attached directly to Si such as phenyl or aralkyl groups such 2-phenylpropyl, in addition to long chain alkyl groups. Waxy cyclopolysiloxanes containing aralkyl groups, that is, silicon-bonded substituents of the formula X-Ph, wherein X denotes a divalent aliphatic organic group bonded to silicon through a carbon atom and Ph denotes an optionally substituted aromatic group, for example 2-phenylpropyl, benzyl, 2-phenylethyl or 2-(t-butylphenyl)ethyl, are particularly preferred. Such aralkyl groups may for example be present in 10 to 80%, preferably 20 to 50% of the siloxane units of the waxy cyclopolysiloxane, usually as methyl aralkyl siloxane units.

The liquid silicones suitable for use in the invention are generally polysiloxanes, for example polydiorganosiloxanes or branched liquid polysiloxanes. Particularly preferred polysiloxanes are those containing aryl, for example phenyl, or aralkyl, for example benzyl, 2-phenylethyl or 2-phenylpropyl groups in addition to alkyl groups such as methyl. The polydiorganosiloxane can be linear or cyclic; cyclic siloxanes such as tetra(2-phenylpropyl)tetramethylcyclotetrasiloxane may be preferred, particularly for use with silicone waxes. The liquid polysiloxane can contain functional groups, for example it can contain hydroxyl groups such as terminal silanol groups in a linear polydiorganosiloxane such as polydimethylsiloxane, alkoxy groups such as methoxy, ethoxy or propoxy bonded to silicon, or amino, amido, alcohol or alkoxy groups substituted in an organic group bonded to silicon.

The waxy hydrophobic mixture of the wax and the liquid silicone is preferably a solid, for example it preferably has a melting point in the range 10-200° C., but can alternatively be a viscous liquid. The liquid silicone can for example be used at up to 100% or even higher based on the weight of wax, such as up to 200 or 300%, particularly if the blend of wax and liquid silicone is solid at 10° C., although the liquid silicone is preferably used at 1 to 60%, most preferably 10 to 30%, based on the weight of wax.

Organic waxes used for delayed release of perfumes have the disadvantage of being quite occlusive. The few fragrance notes released upon time are hardly perceivable, in particular the ones with a high odour threshold. Silicone waxes are less occlusive but are more expensive. Blending either sort of wax with silicone fluid changes the release pattern. The formulator has now access to as many release profiles as he wants according to the characteristics of the perfume and the application without needing a new wax for each.

We have found that the addition of a silicone fluid to an organic wax tends to increase the rate of release, which makes the notes more easily perceivable. Thus according to one aspect of the invention the silicone liquid reduces the occlusivity of the organic wax.

On the other hand, we have found that the addition of a silicone fluid to a silicone wax tends to decrease the rate of release of fragrance from the composition, which is of interest for highly volatile notes or to reduce the level of silicone wax without changing the release profile. Thus according to another aspect of the invention the silicone liquid decreases the rate of release of fragrance from a composition containing silicone wax.

The wax can be a blend of two or more waxes if they are compatible. For example, it can be a blend of an organic wax and a silicone wax. An example of such a blend is a mixture of trimethylstearyloxysilane with octadecanol, sold under the Trade Mark ‘Dow Corning 580’. In general, the silicone liquid tends to reduce the occlusivity of such a blend if the blend contains more organic wax than silicone wax but tends to decrease the rate of release of fragrance if the composition contains more silicone wax than organic wax.

In one preferred form of the invention the composition for controlled release of fragrance forms the disperse phase of an oil-in-water emulsion. Most preferably, the continuous phase comprises an aqueous solution of concentration at least 0.1 molar of a salt capable of ionic disassociation in water. We have found that the high ionic strength of the continuous phase increases the partition coefficient between the continuous phase and the hydrophobic waxy matrix, so that the fragrance tends to stay in the wax phase rather than diffusing into the continuous phase.

The salt present in the continuous phase can for example be an alkali metal, ammonium or alkaline earth metal salt. It can be an inorganic salt such as a chloride, sulphate or phosphate but is preferably an organic salt, particularly a carboxylate. The salt can be a monocarboxylate such as an acetate or propionate, for example sodium acetate, or a di- or poly-carboxylate salt, for example a succinate, phthalate or citrate. The salt can be a polyelectrolyte, for example a salt of a polymeric acid such as a polycarboxylate, e.g. a polyacrylate or polymethacrylate or a salt of an acrylic or methacrylic acid copolymer. Examples of such polyelectrolyte salts are sold under the Trade Mark ‘Sokolan’. The salt in the continuous phase can alternatively be a salt of a polycation such as a polymer having pendant quaternary ammonium groups. An example of such a cationic polymer is sold under the Trade Mark ‘Merquat’ and contains dimethyl diallyl ammonium chloride, methacrylamidopropyl trimethyl ammonium chloride or N,N-dimethylimidazolinium groups. The salt preferably has no surfactant properties; in general, the salt should not contain any organic group which has a chain of 8 or more carbon atoms unsubstituted by polar groups. The concentration of the salt in the aqueous solution which forms the continuous phase of the emulsion is preferably at least 0.1 M (molar), more preferably at least 1 M, up to 5 or 10 M. In the case of a salt of a polyelectrolyte, the concentration is measured as the concentration of the non-polymeric ion of the salt.

The emulsion can conveniently be formed by melting the blend of a fragrance composition, wax and liquid silicone and emulsifying it in the continuous phase using at least one surfactant. The surfactant is preferably immiscible with the said blend. The surfactant can be a cationic, anionic, nonionic or amphoteric surfactant, although the ionic surfactants are more likely to be immiscible with the perfume wax blend. Cationic surfactants are particularly preferred because of their propensity to adsorb at surfaces, in particular onto fabrics. Examples of suitable cationic surfactants include alkylamine salts, quaternary ammonium salts, sulphonium salts and phosphonium salts.

The emulsion can alternatively be made by emulsifying the wax and liquid silicone in the absence of perfume. The fragrance composition is post-added to the emulsion, which is then heated above the melting point of the waxy hydrophobic material and left standing at this temperature, preferably for a period of at least 10 minutes, for example 30-60 minutes, allowing the perfume to diffuse within the droplet of hydrophobic waxy material.

The composition for controlled release of fragrance can be produced in various forms. For some applications the controlled release fragrance composition can simply be mixed with a cleaning or cosmetic composition. The controlled release fragrance composition can be produced in particulate form, which may be preferred for blending with a solid cleaning product such as a powder detergent. An emulsion as described above can be deposited on a particulate solid carrier or can be spray dried. Alternatively the blend of fragrance composition, wax and liquid silicone can be melted and the melt can be deposited on a particulate solid carrier or can be spray dried. Examples of suitable solid carriers include soda ash (sodium carbonate), zeolites and other aluminosilicates or silicates, for example magnesium silicate, phosphates, for example powdered or granular sodium tripolyphosphate, sodium sulphate, sodium carbonate, sodium perborate, cellulose derivatives such as sodium carboxymethylcellulose, granulated or native starch and clay.

The carrier particles are preferably mixed while being treated in a granulation process which produces agglomerated granules. In one preferred process, the particles are agitated in a vertical, continuous high shear mixer in which an emulsion of the composition for controlled release of fragrance is sprayed onto the particles. If needed to improve the granulation process, the emulsion can be diluted with for example water, molten polyethylene glycol or an aqueous solution of polyelectrolyte. One example of such a mixer is a Flexomix mixer supplied by Hosokawa Schugi. The spraying and mixing produces agglomerated granules. Alternative mixers may be used, for example horizontal mixers such as pin mixers or paddle mixers, ploughshare mixers, twin counter-rotating paddle mixers, or intensive mixers including a high shear mixing arm within a rotating cylindrical vessel. Alternatively a fluid bed coating procedure can be used. Advantageously a process of granulation by mixing can be followed by cooling and drying in a continuous fluid bed.

Granules produced from an emulsion whose continuous phase is an aqueous solution of a polyelectrolyte salt may be post-coated with a material, for example a polymer, of opposite charge to the polyelectrolyte. If the salt in the continuous phase of the emulsion is a cationic polyectrolyte salt, for example, the granules can be post-coated with an anionic polyelectrolyte. Such post-coating may improve the deposition of the perfume on a fabric which is subsequently washed or rinsed in the presence of the granules.

Granules with a perfume content of up to 15%, for example 8-12%, by weight can readily be produced by the process of the invention. An emulsion according to the invention can have a perfume content of up to 30 or 40% or even 50% by weight.

In an alternative process according to the invention for producing a fragrant powdered cleaning product, the emulsion described above is deposited on a powdered cleaning product, for example by spraying the emulsion onto a detergent powder composition, and is subsequently dried.

In a process according to the invention for producing a fragrant liquid cleaning product, for example a liquid laundry detergent, household cleaning product, fabric softener, hair shampoo or soap or shower gel for personal washing, or a roll-on or spray deodorant, an emulsion as described above is dispersed in the liquid cleaning product, or the blend of a fragrance composition, wax and liquid silicone can be emulsified in the liquid cleaning product. When producing a cleaning product or personal care product in gel form, for example a stick deodorant, an emulsion as described above can be incorporated in the product when it is in liquid form, or the blend of a fragrance composition, wax and liquid silicone can be emulsified in the product when it is in liquid form, before it is gelled. A tumble drier sheet can be produced by impregnating a textile material with an emulsion as described above.

The delayed release fragrance composition of the invention can alternatively be applied as a coating to a substrate to give sustained release of perfume from the surface. The coating can for example be an emulsion as described above.

The invention is illustrated by the following Examples:

### EXAMPLE 1

5 g of benzaldehyde (a fragrance), 10 g of a waxy fatty acid sold under the Trade Mark ‘Radiacid 0068’ and 5 g of a liquid phenyl(trimethylsiloxy)silane sold under the Trade Mark ‘Dow Corning DC 556’ were blended at a temperature above the melting point of the waxy fatty acid and were allowed to cool. The blend was then placed in an oven at 35° C. for a weight loss assessment.

In a comparative experiment 1a, 5 g of benzaldehyde (BZA) and 10 g of Radiacid 0068 were blended and placed in an oven at 35° C. for a weight loss assessment. The results are shown in Table 1 below

TABLE 1

| | | | Time (days) | | | |
|---|---|---|---|---|---|---|
| Systems | Ratio | | 1 | 2 | 8 | 15 |
| Example 1a BZA/wax | 1/2 | Residual | 99.3 | 98.6 | 92.4 | 82.6 |
| Example 1 BZA/wax/ | 1/2/1 | perfume % | 95.1 | 90.7 | 77.9 | 66.7 |
| DC 556 | | | | | | |

As can be seen from Table 1, the waxy fatty acid alone delays release of the fragrance, but largely occludes the fragrance so that much of it is never released. The liquid silicone decreases the high occlusivity of the fatty acid, increasing the odour perception.

### EXAMPLE 2

A silicone wax of melting point 66° C. was prepared by reacting an olefin mixture consisting predominantly of C26 and C28 olefins with tetramethylcyclotetrasiloxane (cyclic SiH compound). 16 g of the silicone wax was melted and blended with 4 g benzaldehyde and 8 g DC556 liquid silicone. The blend was then placed in an oven at 35° C. for a weight loss assessment.

### EXAMPLE 3

5 g of benzaldehyde, 10 g of the silicone wax of Example 2 and 10 g of DC 556 ere weighed in an open cup and placed in an oven at 35° C. for a weight loss assessment.

In comparative experiments 2a and 2b, 16 g and 24 g respectively of the silicone wax were blended with 4 g benzaldehyde and placed in an oven at 35° C. for a weight loss assessment. The results are shown in Table 2 below

TABLE 2

| | | | Time (days) | | | |
|---|---|---|---|---|---|---|
| Systems | Ratios | | 1 | 2 | 8 | 15 |
| Example 2a BZA/Si | 1/4 | Residual | 87.4 | 68.9 | 8.7 | 3.9 |
| wax | | | | | | |
| Example 2b BZA/Si | 1/6 | perfume % | 96.0 | 83.8 | 31.8 | 14.1 |
| wax | | | | | | |
| Example 2 BZA/Si | 1/2/2 | | 99.0 | 85.8 | 27.8 | 6.2 |
| wax/DC 556 | | | | | | |
| Example 3 BZA/Si | 1/4/2 | | 98.0 | 94.7 | 52.7 | 24.5 |
| wax/DC 556 | | | | | | |

The results shown in Table 2 clearly demonstrate that when DC556 liquid silicone is blended with a waxy silicone it provides a longer lasting effect, which is not reached when using only the waxy silicone. DC 556 alone brings no benefit; more than 80% of the benzaldehyde is lost within 1 day.

### EXAMPLE 4

A liquid silicone consisting mainly of tetra(2-phenylpropyl)tetramethylcyclotetrasiloxane was prepared by reacting alpha-methylstyrene with tetramethylcyclotetrasiloxane. 10 g of the silicone wax of Example 2 was melted and blended with 5 g benzaldehyde and 10 g of the liquid silicone prepared above. The blend was then placed in an oven at 35° C. for a weight loss assessment. Comparative blends 4a and 4b containing benzaldehyde and the silicone wax in weight ratio 1:2 and 1:4 respectively with no liquid silicone were tested similarly. The results are shown in Table 3.

TABLE 3

| | | | Time (days) | | | | |
|---|---|---|---|---|---|---|---|
| Systems | Ratios | | 1 | 2 | 6 | 8 | 15 |
| Example 4a | 1/2 | Residual | 88.7 | 64.2 | 6.1 | 4.2 | 3.6 |
| BZA/Si wax | | | | | | | |
| Example 4b | 1/4 | perfume % | 87.4 | 68.9 | 15.6 | 8.7 | 3.9 |
| BZA/Si wax | | | | | | | |
| Example 4 | 1/2/2 | | 94.2 | 79.9 | 43.6 | 31.5 | 14.4 |
| BZA/Si | | | | | | | |
| wax/liquid | | | | | | | |

The results shown in Table 3 clearly demonstrate that when the liquid tetra(2-phenylpropyl)tetramethylcyclotetrasiloxane is used to replace partly the silicone wax, a longer lasting delayed release of fragrance is achieved. When tetra(2-phenylpropyl)tetramethylcyclotetrasiloxane is used without wax, it provides no controlled release benefit (results similar to benzaldehyde alone).

1. A composition for controlled release of fragrance comprising a blend of a fragrance composition and a waxy hydrophobic material, characterised in that the waxy hydrophobic material is a mixture of a wax and a liquid silicone compatible with the wax.

2. A composition according to claim 1 characterised in that the wax is an organic wax containing no silicone in its molecule.

3. A composition according to claim 2 characterised in that the wax is a fatty acid or ester thereof

4. A composition according to claim 2 characterised in that the wax is microcrystalline wax, paraffin wax, or polyethylene wax.

5. A composition according to claim 1 characterised in that the wax is a polysiloxane.

6. A composition according to claim 5 characterised in that at least 20% of the silicon atoms in the polysiloxane wax have an alkyl substituent having 16 to 100 carbon atoms.

7. A composition according to claim 5 characterised in that the polysiloxane wax also contains aryl or aralkyl substituents.

8. A composition according to claim 1 characterised in that the liquid silicone is a polysiloxane containing aryl or aralkyl substituents.

9. A composition according to claim 1 characterised in that the liquid silicone is a cyclopolysiloxane.

10. A composition according to claim 1 characterised in that the liquid silicone is present at 1 to 100% by weight based on the wax.

11. A composition according to claim 1 characterised in that the waxy hydrophobic mixture has a melting point in the range 10-200° C.

12. A composition according to claim 11 in particulate form.

13. A composition according to claim 1, characterised in that the blend of fragrance composition and waxy hydrophobic material is present as the disperse phase of an oil-in-water emulsion.

14. A composition according to claim 13, characterised in that the blend of fragrance composition and waxy cyclopolysiloxane is dispersed dispersed in a continuous phase comprising an aqueous solution of concentration at least 0.1 molar of a salt capable of ionic disassociation in water.

15. A process for the preparation of a composition for controlled release of fragrance characterised in that a molten wax is blended with a fragrance composition and a liquid silicone which is compatible with the wax.

16. A process for the preparation of an emulsion as defined in claim 14, characterised in that the blend of wax, fragrance composition and liquid silicone prepared according to claim 15 is emulsified in an aqueous solution of the salt.

17. A process for the preparation of an emulsion as defined in claim 13, characterised in that the wax and liquid silicone are emulsified in an aqueous medium in the absence of the fragrance composition, the fragrance composition is added to the resulting emulsion, and the emulsion is then heated above the melting point of the waxy hydrophobic mixture of wax and silicone and held at a temperature above the melting point of the waxy hydrophobic mixture to allow the fragrance composition to diffuse within the droplets of the waxy hydrophobic mixture.

18. (canceled)
